(19) United States
(12) Patent Application Publication
(10) Pub. No.: US 2026/0160751 A1
(43) Pub. Date: Jun. 11, 2026

(54) BREATH SENSING APPARATUS

(71) Applicant: Asahi Kasei Microdevices Corporation, Tokyo (JP)

(72) Inventors: Naoya UGOMORI, Tokyo (JP); Shota ISSHIKI, Tokyo (JP)

(21) Appl. No.: 19/411,333

(22) Filed: Dec. 7, 2025

(30) Foreign Application Priority Data
Dec. 10, 2024 (JP) 2024-215511
Oct. 10, 2025 (JP) 2025-172149

(51) Int. Cl. G01N 33/497; B60Q 9/00; B60R 16/033

(52) U.S. Cl. CPC G01N 33/4972; B60Q 9/00; B60R 16/033

(57) ABSTRACT

There is provided a breath sensing apparatus which senses alcohol contained in exhaled breath in an operator's cabin of a moving body, the breath sensing apparatus including: a sensing unit which senses the alcohol; and a communication unit which communicates with an external device of the moving body, in which the communication unit receives a result request from the external device, and transmits a sensing result in the sensing unit to the external device according to the result request. When the communication unit receives the result request, the sensing unit may perform sensing of the alcohol.

The contents of the following patent application(s) are incorporated herein by reference:
NO. 2024-215511 filed in JP on Dec. 10, 2024
NO. 2025-172149 filed in JP on Oct. 10, 2025.

### BACKGROUND

### 1. Technical Field

The present invention relates to a breath sensing apparatus.

### 2. Related Art

Patent Document 1 discloses a “drunk driving prevention system that prevents drunk driving via communication between an external server and an on-vehicle component”.

Patent Document 1: Japanese Patent Application Publication No. 2012-210931

### BRIEF DESCRIPTION OF THE DRAWINGS

FIG. 1 is a schematic diagram showing an example of a moving body 400 equipped with a breath sensing apparatus 300 according to one embodiment of the present invention.

FIG. 2 is a block diagram showing a configuration example of the breath sensing apparatus 300.

FIG. 3 is a block diagram showing another configuration example of the breath sensing apparatus 300.

FIG. 4 is a block diagram showing another configuration example of the breath sensing apparatus 300.

FIG. 5 is a block diagram showing an example of a sensing unit 100.

### DESCRIPTION OF EXEMPLARY EMBODIMENTS

Hereinafter, the present invention will be described through embodiments of the invention, but the following embodiments do not limit the invention according to the claims. Further, not all of combinations of features described in the embodiments are essential to the solving means of the invention.

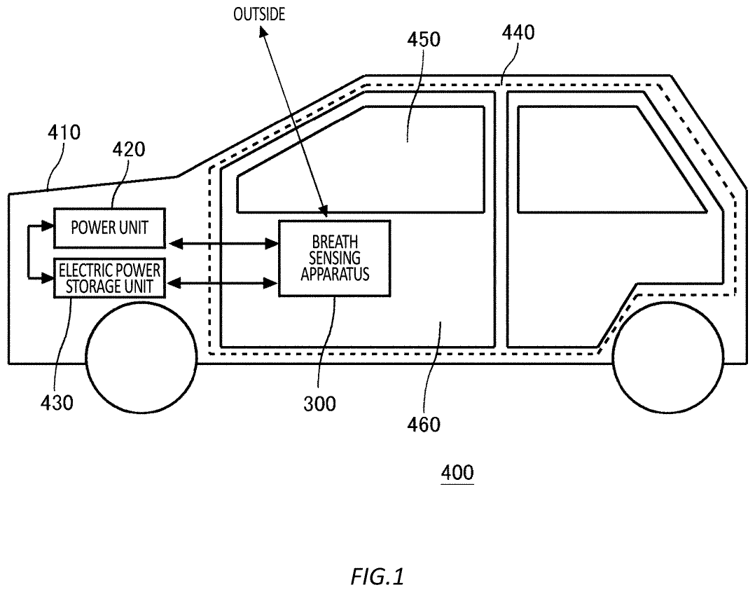

FIG. 1 is a schematic diagram showing an example of a moving body 400 equipped with a breath sensing apparatus 300 according to one embodiment of the present invention. The moving body 400 is, for example, an automobile, but is not limited thereto. The moving body 400 may be a ground moving body such as a vehicle which moves on the ground, may be an airborne moving body such as a flying body which flies through air, may be a waterborne moving body such as a vessel which moves on the water, may be an underwater moving body such as a submersible which moves underwater, or may be a moving body which moves in another location.

The moving body 400 includes an operator's cabin 440 for boarding by an operator who operates the moving body 400. The operator's cabin 440 may have a space for boarding by a passenger other than the operator. The operator's cabin 440 is, for example, a space in which equipment for an operation of the moving body 400, such as a steering wheel in an automobile, is installed. The operator's cabin 440 in the present example is a space surrounded by a moving body housing 410 of the moving body 400. The moving body housing 410 includes, for example, a body portion of an automobile. The moving body housing 410 may include at least one of one or more windows 450 or one or more doors 460. The window 450 and the door 460 may be openable and closable between the operator's cabin 440 and an exterior space. The moving body housing 410 may include, in addition to the window 450 and the door 460, a portion which is openable and closable.

The breath sensing apparatus 300 senses alcohol contained in exhaled breath in the operator's cabin 440 of the moving body 400. The breath sensing apparatus 300 in the present example may be an apparatus which is capable of sensing alcohol without requiring an operation of a device by an occupant such as the operator and without requiring an action such as intentional blowing of breath by the occupant. The breath sensing apparatus 300 senses alcohol contained in the exhaled breath of the occupant such as the operator, by measuring air in the operator's cabin 440. A part or a whole of the breath sensing apparatus 300 is provided in the operator's cabin 440. The breath sensing apparatus 300 may sense a concentration of alcohol (ppm). The breath sensing apparatus 300 may determine whether or not a sensed concentration of alcohol is within an allowable range. When the sensed concentration of alcohol exceeds the allowable range, the breath sensing apparatus 300 may prohibit the operator from operating the moving body 400, may restrict speed and a movement range of the moving body 400, or may restrict a human operation to switch to an autonomous driving mode. The breath sensing apparatus 300 may estimate a blood alcohol concentration of the operator, based on the concentration of alcohol contained in the sensed exhaled breath. When the sensed concentration of alcohol exceeds the allowable range, the breath sensing apparatus 300 may present the operator with an alarm including information to that effect; may record the sensed alcohol concentration, a time, the operator, and a location; or may present the operator with a message for remeasuring the alcohol concentration. The breath sensing apparatus 300 may acquire location information of the moving body 400 to change the allowable range of the alcohol concentration based on the acquired location information. For example, in Japan, an alcohol concentration of 0.15 mg/L or higher in the exhaled breath is considered as driving under the influence, whereas in Germany, an alcohol concentration of 0.25 mg/L or higher in the exhaled breath constitutes a legal violation, and thus, the breath sensing apparatus 300 may change the allowable range of the alcohol concentration based on the location information of the moving body 400.

The moving body 400 in the present example includes a power unit 420 and an electric power storage unit 430. The power unit 420 generates power to move the moving body 400. The power unit 420 may be, for example, an internal combustion engine such as an engine which generates power by combusting fuel. The power unit 420 may be, for example, an electric motor such as a motor which rotates according to electric power. When the sensed concentration of alcohol exceeds the allowable range, the breath sensing apparatus 300 may cause power generation by the power unit 420 to be stopped. When the power unit 420 is the electric motor such as the motor, a fuel cell may be included as an electric power source; and when the sensed concentration of alcohol exceeds the allowable range, the breath sensing apparatus 300 may stop an operation of the fuel cell.

The electric power storage unit 430 stores the electric power and supplies the electric power to equipment of the moving body 400. The electric power storage unit 430 may supply the electric power to the power unit 420, may supply the electric power to the breath sensing apparatus 300, or may supply the electric power to other equipment such as an air conditioner that adjusts a temperature in the operator's cabin 440 and a display unit that displays information. When the sensed concentration of alcohol exceeds the allowable range, the breath sensing apparatus 300 may cause electric power supply from the electric power storage unit 430 to be stopped.

The breath sensing apparatus 300 in the present example transmits, according to a result request from an external device of the moving body 400, a sensing result of alcohol to the external device. The external device which transmits the result request and the external device which receives the sensing result may be the same device, or may be devices different from each other. The breath sensing apparatus 300 is controlled from the outside of the moving body 400, thereby making it possible to control, from the outside, a timing of sensing alcohol contained in the exhaled breath of the operator or the like, or a timing of transmitting the sensing result. Therefore, it is possible to perform sensing of a state such as drunk driving, at any timing, regardless of an intention of the occupant or the like.

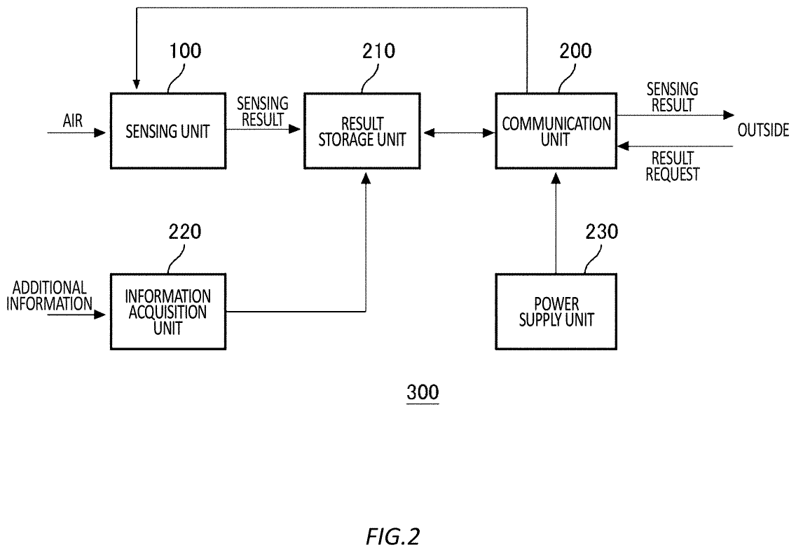

FIG. 2 is a block diagram showing a configuration example of the breath sensing apparatus 300. The breath sensing apparatus 300 includes a sensing unit 100 and a communication unit 200. The breath sensing apparatus 300 may further include some or all of a result storage unit 210, an information acquisition unit 220, and a power supply unit 230.

The sensing unit 100 senses alcohol. The sensing unit 100 may sense a component other than alcohol. For example, the sensing unit 100 may sense the concentration (ppm) of alcohol and the concentration (ppm) of carbon dioxide in the operator's cabin 440. The sensing unit 100 may automatically sense alcohol without depending on the intention of the occupant. The sensing unit 100 may correct the sensed concentration of alcohol based on the sensed concentration of carbon dioxide. In the present specification, the concentration sensed by the sensing unit 100 may be referred to as the sensed concentration. The sensing unit 100 may have a sensor of a non-dispersive infrared absorption type (NDIR type) or an electrochemical type (fuel cell type).

The communication unit 200 communicates with the external device of the moving body 400. The communication unit 200 receives the result request from the external device. The communication unit 200 transmits the sensing result in the sensing unit 100 to the external device according to the result request. The communication unit 200 may automatically transmit the sensing result to the external device which has transmitted the corresponding result request. In another example, the communication unit 200 may transmit the sensing result to an external device different from the external device which has transmitted the result request. The sensing result of the sensing unit 100 may include information of the sensed concentration of alcohol, and may include information regarding whether or not the sensed concentration of alcohol exceeds a reference value.

When the result request is received, the communication unit 200 transmits the sensing result of alcohol to the external device. When the result request is received, the communication unit 200 may activate the sensing unit 100 and cause it to sense alcohol. When the result request is received, the communication unit 200 may acquire a past sensing result that has been already stored in the result storage unit 210, and transmit the past sensing result to the external device. For example, the communication unit 200 may transmit, to the external device, a history of the sensing result of alcohol in a predetermined period in the past based on a timing when the result request is received. With such a configuration, it is possible to control, from the outside, a timing of sensing alcohol contained in the exhaled breath of the operator or the like, or a timing of acquiring the sensing result.

The communication unit 200 may communicate with the external device by wireless communication. The communication unit 200 may have, in the moving body housing 410, such as a window 450, an antenna for communicating with the external device. In a case where a mobile terminal is arranged in a vicinity of the antenna, the communication unit 200 may communicate with the mobile terminal. For example, the communication unit 200 may communicate with the mobile terminal carried by a police officer or the like. This makes it possible to easily sense a current or past alcohol consumption state of the occupant or the like. In another example, the communication unit 200 may communicate with the external device provided in a non-moving device such as a signal or a speed measuring instrument installed on a road. In this case, even in a state in which the moving body 400 is moving, it is possible to easily communicate between the external device and the communication unit 200. The communication unit 200 may communicate with the external device in a state in which the moving body 400 is moving, or may communicate with the external device in a state in which the moving body 400 is stopped.

The power supply unit 230 supplies source electric power to the communication unit 200. The power supply unit 230 may be capable of supplying the source electric power to the communication unit 200, in a state in which the power unit 420 of the moving body 400 is not activated. The power supply unit 230 may have a power generation element that generates the source electric power by a radio wave or the like received from the external device, or may be a battery that accumulates the electric power supplied from a power source such as the electric power storage unit 430. The power supply unit 230 may supply the source electric power to the configuration element of the breath sensing apparatus 300 other than the communication unit 200, such as the sensing unit 100. The electric power storage unit 430 may supply the source electric power to the breath sensing apparatus 300.

The communication unit 200 may repeatedly and periodically be activated and deactivated. For example, when the moving body 400 is not activated, the communication unit 200 may repeatedly and periodically be activated and deactivated. For each activation, the communication unit 200 may check for a presence or an absence of the result request from the external device. By such an operation, it is possible to suppress electric power consumption by the communication unit 200.

When the communication unit 200 receives the result request, the sensing unit 100 may perform sensing of alcohol. When the result request is received, the communication unit 200 may activate the sensing unit 100. The activation of the sensing unit 100 refers to starting the supply of the source electric power from the power supply unit 230 or the like to the sensing unit 100. In another example, the sensing unit 100 may perform sensing of alcohol at a preset timing. For example, the sensing unit 100 may perform sensing of alcohol by a preset cycle. The sensing unit 100 may be activated and deactivated each time sensing of alcohol is performed. The sensing unit 100 may continuously remain activated while the moving body 400 is activated. In this case, it is not needed to wait for the sensing of alcohol until the sensing unit 100 is stabilized after the activation.

The result storage unit 210 stores the sensing result by the sensing unit 100. The communication unit 200 may transmit, to the external device, the sensing result that is stored by the result storage unit 210. The result storage unit 210 may store each sensing result in association with additional information acquired by the information acquisition unit 220. The additional information includes at least some of an acquisition time of the sensing result by the sensing unit 100, location information of the moving body 400 when the sensing unit 100 acquires the sensing result, speed information of the moving body 400 when the sensing unit 100 acquires the sensing result, or information relating to the occupant of the moving body 400 when the sensing unit 100 acquires the sensing result. The information relating to the occupant may be information for identifying the occupant. For example, the information relating to the occupant may be an image including a face of the occupant. The breath sensing apparatus 300 may acquire an image of the face of the occupant seated in an operator's seat when the sensing unit 100 performs sensing of alcohol. The information relating to the occupant may be identification information such as a name of the occupant, which is determined from the image.

The communication unit 200 may extract the sensing result corresponding to the received result request, from the result storage unit 210, based on the additional information. For example, the acquisition time of the sensing result may be specified in the result request. The communication unit 200 may extract the sensing result in which the acquisition time included in the additional information is closest to the acquisition time that is specified in the result request, and transmit the extracted sensing result to the external device. In the result request, at least one of the acquisition time, the range of the location information, the range of the speed information, or the information relating to the occupant, which are described above, may be specified. The communication unit 200 may extract the additional information closest to the information that is specified in the result request, or the additional information included in the range that is specified in the result request; and transmit the corresponding sensing result. In this manner, the necessary sensing result can be specified from the outside and acquired.

The communication unit 200 may transmit, to the external device, the sensing result stored by the result storage unit 210 in association with the additional information. This makes it possible for the external device to acquire the sensing result in association with the additional information, and makes it possible to analyze a situation at a time when the sensing result is acquired. For example, by acquiring information relating to a driver, information relating to the speed of the moving body 400, and information relating to the location of the moving body 400, when the sensing result exceeding a reference value of the alcohol concentration is acquired, it is possible to analyze in detail a manner of traffic regulation violations by the driver. The external device may display, on a map, the location of the moving body 400 in a drunk driving state. In this manner, enforcement against traffic regulation violations can be performed easily.

When the sensing result stored in the result storage unit 210 satisfies a preset condition, the communication unit 200 may transmit the corresponding additional information in association with the sensing result. For example, when the alcohol concentration indicated by the sensing result is higher than or equal to a reference value, the communication unit 200 may transmit, to the external device, the additional information in association with the sensing result. When the alcohol concentration indicated by the sensing result is lower than the reference value, the communication unit 200 may transmit only the sensing result to the external device without including the additional information. This makes it possible to suppress leakage of personal information included in the additional information.

When the external device which is to receive the sensing result is capable of receiving the sensing result, the communication unit 200 may transmit the sensing result to the external device. The external device which is to receive the sensing result may be an external device which has transmitted the result request, or may be another external device. For example, there may be a case where the moving body 400 is in a state of moving, receives the result request from any external device, activates the sensing unit 100 to acquire the sensing result, and transmits the sensing result to the external device. There may be a case where the moving body 400 moves outside a range in which it is possible to communicate with the external device, at a time of transmitting the sensing result. In such a case, the result storage unit 210 stores the sensing result in association with the result request.

On the condition that the moving body 400 moves into a range in which it is possible to communicate with another external device which is linked to the external device, the communication unit 200 transmits the sensing result stored in the result storage unit 210 to the other external device. The communication unit 200 may transmit the sensing result in association with the result request. The other external device is an external device which is capable of receiving the sensing result corresponding to the result request. The other external device may, for example, transmit the sensing result and the result request to a management server. When the moving body 400 moves again into a range in which it is possible to communicate with the external device which has transmitted the result request, the communication unit 200 may transmit the sensing result in association with the result request. The external device may be, for example, a speed sensor, may be a traffic light, or may be another device. The external device which transmits the result request and the external device which receives the sensing result may be devices having the same function, or may be devices having functions different from each other. For example, the speed sensor may transmit the result request, and the traffic light provided at a different location may receive the sensing result. By such a process, even when the moving body 400 is moving at a high speed, it is possible to acquire and transmit the sensing result according to the request from the external device.

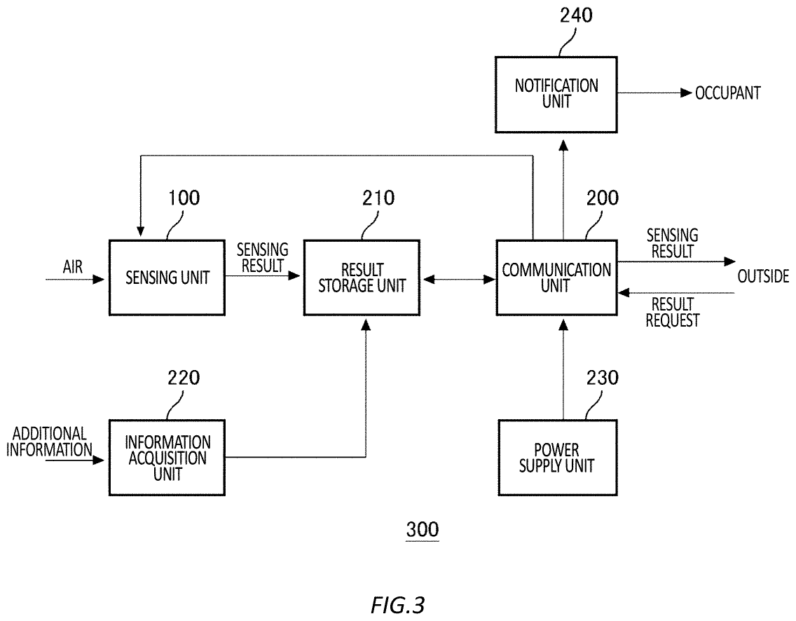

FIG. 3 is a block diagram showing another configuration example of the breath sensing apparatus 300. The breath sensing apparatus 300 in the present example further includes a notification unit 240 with respect to the breath sensing apparatus 300 in another example. The configuration other than the notification unit 240 is similar to that of any breath sensing apparatus 300 described in the present specification.

The notification unit 240 notifies the occupant of the moving body 400 that the communication unit 200 has transmitted the sensing result to the external device. The notification unit 240 may display the notification on a display unit provided in the moving body 400, or may cause the notification to be output by the audio device provided in the moving body 400. The notification unit 240 may transmit the notification to a device such as a mobile terminal or a wearable terminal of the occupant registered previously.

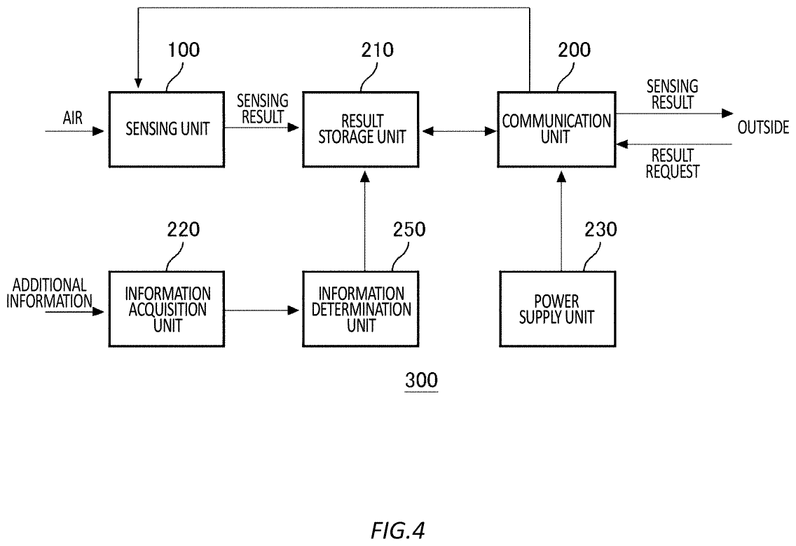

FIG. 4 is a block diagram showing another configuration example of the breath sensing apparatus 300. The breath sensing apparatus 300 in the present example further includes an information determination unit 250 with respect to the breath sensing apparatus 300 in another example. The configuration other than the information determination unit 250 is similar to that of any breath sensing apparatus 300 described in the present specification.

The information acquisition unit 220 in the present example acquires the additional information indicating an open/closed state of the window 450 when the sensing unit 100 acquires the sensing result, in association with the sensing result. The open/closed state may be information indicating, for example, to what extent the window 450 is open. The open/closed state may be acquired from a control unit which controls opening and closing of the window 450. When the opening and closing of the window 450 is controlled by an electrical signal, it is possible to generate the open/closed state based on the electrical signal. When the opening and closing of the window 450 is manually controlled, the moving body 400 may include a position sensor which senses the open/closed state by sensing a position of an end portion or the like of the window 450.

The information determination unit 250 determines whether or not the open/closed state of the window 450 included in the additional information satisfies a predetermined condition. For example, the information determination unit 250 determines whether or not an open area of the window 450 is smaller than or equal to a reference value. The reference value may be one half or less of a maximum area by which the window 450 can be opened, may be ¼ or less, or may even be 0. That is, the information determination unit 250 may determine whether or not the window 450 is closed.

When the window 450 is open, there may be a case where accuracy of the sensing result of alcohol by the breath sensing apparatus 300 deteriorates. When the additional information satisfies a predetermined condition, the communication unit 200 in the present example transmits the corresponding sensing result to the external device. When the additional information does not satisfy the predetermined condition, the communication unit 200 in the present example may not transmit the corresponding sensing result to the external device. This makes it possible to prevent the sensing result with low accuracy from being transmitted to the external device. The communication unit 200 may transmit, together with the sensing result, determination information corresponding to whether or not the additional information satisfies the condition. The determination information may be, for example, information indicating that the accuracy of the corresponding sensing result of alcohol is low.

The information determination unit 250 may determine whether or not the additional information other than the open/closed state of the window 450 satisfies a predetermined condition. The information determination unit 250 may determine whether or not a traveling speed of the moving body 400 is higher than or equal to a predetermined value. When it is determined that the window 450 is open and the traveling speed of the moving body 400 is higher than or equal to the predetermined value, the communication unit 200 may not transmit the corresponding sensing result to the external device. The information determination unit 250 may determine whether or not operation information of the air conditioner satisfies a predetermined condition. For example, the operation information may include an orientation of the air conditioner, a temperature, intensity, an internal air circulation, an outside air intake, and an activation or deactivation state and intensity of seat ventilation.

The result storage unit 210 may store the sensing result in association with a determination result by the information determination unit 250. When the result request is received, the communication unit 200 may extract a newest sensing result among the sensing results determined by the information determination unit 250 to satisfy the condition, and transmit the extracted newest sensing result to the external device. In another example, the result storage unit 210 may store, among the sensing results sensed by the sensing unit 100, only a sensing result determined by the information determination unit 250 to satisfy the condition.

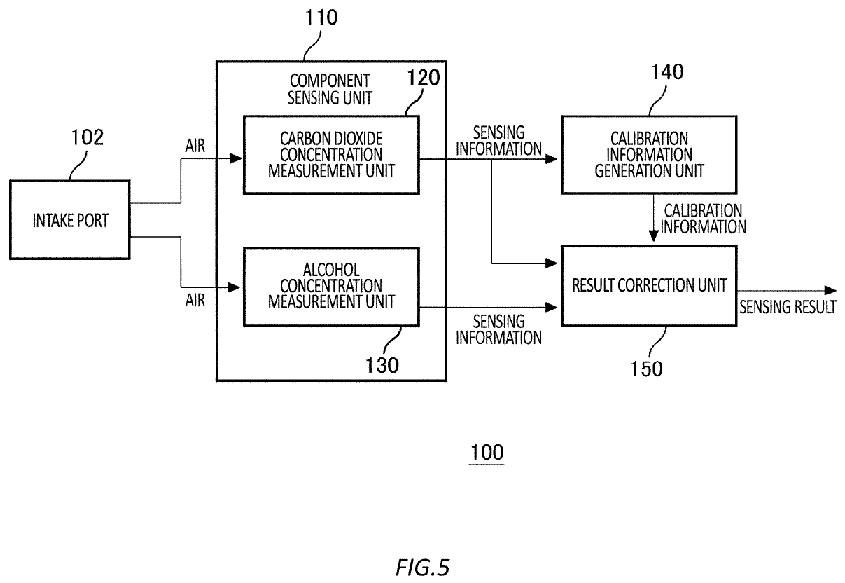

FIG. 5 is a block diagram showing an example of the sensing unit 100. The sensing unit 100 includes a component sensing unit 110, a calibration information generation unit 140, and a result correction unit 150. The sensing unit 100 may further include an intake port 102.

The component sensing unit 110 obtains, through sensing, sensing information indicating the concentrations of alcohol and carbon dioxide contained in the exhaled breath. The component sensing unit 110 may obtain, through sensing, sensing information indicating the concentration of alcohol and sensing information indicating the concentration of carbon dioxide. Each piece of sensing information is a signal, the value of which varies according to a magnitude of the concentration of each target component (alcohol and carbon dioxide in the present example) contained in the exhaled breath. For example, the sensing information is a signal of a value corresponding to intensity of light that has passed through a gas containing the exhaled breath, at a wavelength corresponding to each target component. The intensity of the light is attenuated according to the concentration of each target component contained in the exhaled breath. The sensing information may be a signal obtained by converting the signal of the light into an electrical signal, or may be a signal obtained by performing predetermined signal processing on the electrical signal. The sensing information may include the concentration value itself for each target component.

The component sensing unit 110 in the present example includes a carbon dioxide concentration measurement unit 120 and an alcohol concentration measurement unit 130. The carbon dioxide concentration measurement unit 120 outputs the sensing information corresponding to the concentration of carbon dioxide (ppm) that is contained in air in the operator's cabin 440 and that is introduced into the component sensing unit 110 via the intake port 102. The carbon dioxide concentration measurement unit 120 is, for example, a sensor of a photoacoustic type, a sensor of a solid electrolyte type, or a sensor of the non-dispersive infrared absorption type (NDIR type), but is not limited thereto.

The alcohol concentration measurement unit 130 outputs the sensing information corresponding to the concentration of alcohol (ppm) that is contained in air in the operator's cabin 440 and that is introduced into the component sensing unit 110 via the intake port 102. The alcohol concentration measurement unit 130 is, for example, a sensor of an electrochemical type (fuel cell type), a sensor of the non-dispersive infrared absorption type (NDIR type), or a sensor of the photoacoustic type, but is not limited thereto. The sensor of an electrochemical type senses, for example, an electric current that is generated by alcohol contained in air.

The calibration information generation unit 140 generates calibration information for calibrating the concentration of carbon dioxide, based on the sensing information of carbon dioxide obtained through multiple sensing by the component sensing unit 110. The calibration information is information for converting a value of each piece of sensing information into the concentration of each target component. The calibration information may be a calibration curve that indicates a relationship between a value of the sensing information and the concentration of the target component. When the sensing information includes the concentration value itself for each target component, the calibration information may be information for correcting the concentration value in the sensing information. In the present specification, the concentration that is calculated from the value of the sensing information by using the calibration information, may be referred to as a “calibrated concentration”. For example, the calibration information may include a gain value that is multiplied by the value of the sensing information to calculate the calibrated concentration, may include a function that calculates the calibrated concentration using the value of the sensing information as a variable, or may include a table in which the value of the sensing information is associated with the calibrated concentration.

An initial value of the calibration information may be preset in the calibration information generation unit 140. The initial value of the calibration information may be set by a manufacturer, a user, or the like of the breath sensing apparatus 300. The calibration information generation unit 140 may update the calibration information, based on the sensing information of carbon dioxide obtained through multiple sensing by the component sensing unit 110. In the present specification, an update of the calibration information may be referred to as a generation of the calibration information.

Measurement accuracy of the carbon dioxide concentration measurement unit 120 may change over time. For example, in the sensor of the NDIR type, a characteristic of a light source, a light guide unit, or a light receiving element changes over time, thereby causing a deviation in the value of the sensing information that is generated by the light receiving element, even when carbon dioxide of the same concentration is measured. Therefore, the deviation may be caused in the calibrated concentration obtained through the calibration, even when carbon dioxide of the same concentration is measured. The calibration information generation unit 140 generates or updates the calibration information so that the deviation in the calibrated concentration obtained through the calibration remains small, even when the value of the sensing information for carbon dioxide of the same concentration changes over time.

When a difference between the calibrated concentrations for predetermined sensing information before and after updating the calibration information exceeds a reference value, the breath sensing apparatus 300 may perform error handling because there is a possibility that the breath sensing apparatus 300 is not operating normally. The reference value may be 500 ppm, may be 100 ppm, or may be another value. The error handling may be a correction of the calibration information so that the difference becomes lower than or equal to the reference value, may be a reacquisition of the calibration information, or may be a notification of an abnormal state to the operator.

From among pieces of sensing information of carbon dioxide obtained through multiple sensing, the calibration information generation unit 140 may extract the sensing information, the corresponding concentration of which has a minimum value. In the present specification, a relative magnitude relationship between the corresponding concentrations may be described as a relative magnitude relationship between pieces of sensing information. For example, among a plurality of pieces of sensing information, the sensing information, the corresponding concentration of which has the minimum value may be referred to as minimum sensing information. The calibration information generation unit 140 may adjust the aforementioned calibration information so that the minimum sensing information is converted into a preset reference concentration. Adjusting the calibration information may be adjusting the aforementioned gain value, may be adjusting each coefficient of a function, or may be updating a table. For example, the calibration information generation unit 140 may calculate the gain value by dividing the reference concentration by a concentration corresponding to the minimum sensing information. For example, the reference concentration may be equivalent to an average carbon dioxide concentration in outside air. The reference concentration may be 400 ppm, or may be another value.

The calibration information generation unit 140 may generate the calibration information, by using a plurality of pieces of sensing information measured within a most recent reference period, among pieces of sensing information measured in the past. The reference period may be preset by a manufacturer or a user of the breath sensing apparatus 300. The reference period may be one week or longer, may be two weeks or longer, or may be one month or longer. The reference period may be six months or shorter, or may be two months or shorter.

The carbon dioxide concentration in the operator's cabin 440 fluctuates due to the exhaled breath of the operator or a passenger. Meanwhile, the carbon dioxide concentration in the operator's cabin 440 is not lower than a carbon dioxide concentration in outside air. Accordingly, it can be estimated that the sensing information having smaller values resulted from measurements taken in a state closer to the carbon dioxide concentration in outside air. Therefore, by adjusting the calibration information so that the minimum sensing information among a plurality of pieces of sensing information is converted into a reference concentration, it is possible to generate the calibration information with comparatively high accuracy.

A number of a plurality of pieces of sensing information used to generate the calibration information may be 10 or more, may be 100 or more, or may be 1000 or more. A method for generating the calibration information is not limited to this method. The calibration information generation unit 140 may use a statistical value obtained from a plurality of pieces of sensing information, selected in ascending order of their values, from among the plurality of pieces of sensing information. The statistical value refers to information obtained by performing statistical processing on a plurality of pieces of sensing information, and the statistical processing may be generating at least one of an average value, a maximum value, a minimum value, a variance, a moment, or a histogram.

The result correction unit 150 corrects the sensing result of alcohol based on the calibrated concentration of carbon dioxide obtained through the calibration with the calibration information. The sensing result of alcohol is, for example, the concentration of alcohol. The result correction unit 150 corrects the concentration of alcohol in a measurement target with the calibrated concentration of carbon dioxide measured concurrently with the concentration of the alcohol.

For example, the result correction unit 150 calculates a degree of dilution of air that has reached the component sensing unit 110, based on the calibrated concentration of carbon dioxide. The degree of dilution is an index indicating how much the exhaled breath of the operator has been diluted until it reaches the component sensing unit 110 from the operator. The degree of dilution may be a value obtained by dividing a preset standard concentration of carbon dioxide by the calibrated concentration of carbon dioxide. For the standard concentration of carbon dioxide, an average value of concentrations of carbon dioxide contained in the exhaled breath of adults may be used, or it may be a value actually measured from the exhaled breath of the operator. The standard concentration of carbon dioxide is, for example, a value in a range from 1% to 9%. The standard concentration of carbon dioxide may be, for example, 3%. The standard concentration of carbon dioxide may be set by a manufacturer or a user of the sensing unit 100.

The result correction unit 150 may calculate the corrected concentration of alcohol by multiplying the concentration of alcohol by the aforementioned degree of dilution. For example, when the degree of dilution is calculated as 150 times based on the calibrated concentration of carbon dioxide, the result correction unit 150 calculates the corrected concentration of alcohol by multiplying the concentration of alcohol by 150. This makes it possible to estimate the concentration of alcohol contained in the exhaled breath of the operator. In another example, the result correction unit 150 may correct a threshold concentration for comparison with the concentration of alcohol, based on the degree of dilution. For example, when the degree of dilution is 150 times, the result correction unit 150 may correct the sensing result of alcohol by dividing the threshold concentration by 150.

The sensing unit 100 in the present example calculates, from the carbon dioxide concentration, the degree of dilution of air measured by the component sensing unit 110, with respect to the exhaled breath of the operator; and corrects the sensing result of alcohol. Therefore, the exhaled breath of the operator may not be directly blown into the component sensing unit 110. The sensing unit 100 in the present example can measure the alcohol concentration of the operator, even in a state in which the operator does not have an intention to measure the alcohol concentration. Then, the calibration information for the carbon dioxide concentration is generated based on the carbon dioxide concentrations (the sensing information in the present example) obtained through multiple measurements inside the operator's cabin 440, and thus it is possible to correct a change over time in a characteristic of the component sensing unit 110 to calculate the degree of dilution with high accuracy, and it is possible to measure the alcohol concentration with high accuracy.

The present example has described an example in which the component sensing unit 110 which senses alcohol contained in the exhaled breath obtains, through sensing, the sensing information indicating the concentrations of alcohol and carbon dioxide contained in the exhaled breath. In another example, the component sensing unit 110 may be configured to obtain, through sensing, the sensing information indicating the concentration of alcohol and the concentration of oxygen. The sensing unit 100 can use the concentration of oxygen instead of the concentration of carbon dioxide in calculating the degree of dilution. In this case, carbon dioxide in the present specification may be read as oxygen. For example, the sensing unit 100 may assume an average oxygen concentration in the atmosphere to be 21%, perform the calibration based on this average oxygen concentration, and calculate a dilution ratio of the exhaled breath based on the calibrated concentration of oxygen. In yet another example, the component sensing unit 110 may obtain, through sensing, sensing information indicating the concentrations of alcohol, carbon dioxide, and oxygen contained in the exhaled breath. In this case, the component sensing unit 110 includes an alcohol concentration measurement unit, a carbon dioxide concentration measurement unit, and an oxygen concentration measurement unit. The component sensing unit 110 may calculate the degree of dilution of the exhaled breath, based on a first degree of dilution calculated based on the concentration of carbon dioxide and a second degree of dilution calculated based on the concentration of oxygen. For example, the sensing unit 100 may use an average value of the first degree of dilution and the second degree of dilution as the degree of dilution of the exhaled breath. By combining a carbon dioxide concentration measurement and an oxygen concentration measurement, it is possible to calculate a more accurate dilution ratio of the exhaled breath.

While the embodiments of the present invention have been described, the technical scope of the present invention is not limited to the above-described embodiments. It is apparent to persons skilled in the art that various alterations or improvements can be added to the above-described embodiments. It is also apparent from the claims that the embodiments added with such alterations or improvements can be included in the technical scope of the present invention.

The operations, procedures, steps, and stages or the like of each process performed by an apparatus, system, program, and method shown in the claims, specification, or drawings can be performed in any order as long as the order is not indicated by “prior to,” “before,” or the like and as long as the output from a previous process is not used in a later process. Even if the process flow is described using phrases such as “first” or “next” in the claims, specification, and drawings, it does not necessarily mean that the process must be performed in this order.

What is claimed is:

1. A breath sensing apparatus which senses alcohol contained in exhaled breath in an operator's cabin of a moving body, the breath sensing apparatus comprising:
a sensing unit which senses the alcohol; and
a communication unit which communicates with an external device of the moving body, wherein
the communication unit receives a result request from the external device, and transmits a sensing result in the sensing unit to the external device according to the result request.

2. The breath sensing apparatus according to claim 1, wherein
when the communication unit receives the result request, the sensing unit performs sensing of the alcohol.

3. The breath sensing apparatus according to claim 2, wherein
when the result request is received, the communication unit activates the sensing unit.

4. The breath sensing apparatus according to claim 1 further comprising:
a result storage unit which stores the sensing result by the sensing unit in association with additional information including at least some of an acquisition time of the sensing result, location information of the moving body when the sensing result is acquired, speed information of the moving body when the sensing result is acquired, or information relating to an occupant of the moving body when the sensing result is acquired, wherein
the communication unit transmits the sensing result in association with the additional information.

5. The breath sensing apparatus according to claim 2 further comprising:
a result storage unit which stores the sensing result by the sensing unit in association with additional information including at least some of an acquisition time of the sensing result, location information of the moving body when the sensing result is acquired, speed information of the moving body when the sensing result is acquired, or information relating to an occupant of the moving body when the sensing result is acquired, wherein
the communication unit transmits the sensing result in association with the additional information.

6. The breath sensing apparatus according to claim 3 further comprising:
a result storage unit which stores the sensing result by the sensing unit in association with additional information including at least some of an acquisition time of the sensing result, location information of the moving body when the sensing result is acquired, speed information of the moving body when the sensing result is acquired, or information relating to an occupant of the moving body when the sensing result is acquired, wherein
the communication unit transmits the sensing result in association with the additional information.

7. The breath sensing apparatus according to claim 4, wherein
when the sensing result satisfies a preset condition, the communication unit transmits the additional information in association with the sensing result.

8. The breath sensing apparatus according to claim 1, further comprising:
a power supply unit which supplies source electric power to the communication unit, in a state in which a power unit of the moving body is not activated.

9. The breath sensing apparatus according to claim 2, further comprising:
a power supply unit which supplies source electric power to the communication unit, in a state in which a power unit of the moving body is not activated.

10. The breath sensing apparatus according to claim 3, further comprising:
a power supply unit which supplies source electric power to the communication unit, in a state in which a power unit of the moving body is not activated.

11. The breath sensing apparatus according to claim 1, further comprising:
a notification unit which notifies an occupant of the moving body that the communication unit has transmitted the sensing result.

12. The breath sensing apparatus according to claim 2, further comprising:
a notification unit which notifies an occupant of the moving body that the communication unit has transmitted the sensing result.

13. The breath sensing apparatus according to claim 3, further comprising:
a notification unit which notifies an occupant of the moving body that the communication unit has transmitted the sensing result.

14. The breath sensing apparatus according to claim 1, further comprising:
a result storage unit which stores the sensing result by the sensing unit, wherein
when the external device is capable of receiving the sensing result, the communication unit transmits the sensing result to the external device.

15. The breath sensing apparatus according to claim 2, further comprising:
a result storage unit which stores the sensing result by the sensing unit, wherein
when the external device is capable of receiving the sensing result, the communication unit transmits the sensing result to the external device.

16. The breath sensing apparatus according to claim 3, further comprising:
a result storage unit which stores the sensing result by the sensing unit, wherein
when the external device is capable of receiving the sensing result, the communication unit transmits the sensing result to the external device.

17. The breath sensing apparatus according to claim 14, wherein
the result storage unit stores the sensing result in association with the result request, and
when the external device is capable of receiving the sensing result corresponding to the result request, the communication unit transmits the corresponding sensing result to the external device.

18. The breath sensing apparatus according to claim 1, wherein
the communication unit transmits the sensing result to the external device which has transmitted the result request.

19. The breath sensing apparatus according to claim 2, wherein
the communication unit transmits the sensing result to the external device which has transmitted the result request.

20. The breath sensing apparatus according to claim 1, wherein
the moving body includes a window which is openable and closable,
the breath sensing apparatus further comprises an information acquisition unit which acquires additional information indicating an open/closed state of the window in association with the sensing result, and
when the additional information satisfies a predetermined condition, the communication unit transmits the sensing result corresponding to the additional information.
